(19) United States
(12) Patent Application Publication
(10) Pub. No.: US 2024/0246892 A1
(43) Pub. Date: Jul. 25, 2024

(54) AZEOTROPIC OR QUASI-AZEOTROPIC COMPOSITION COMPRISING CHLOROTRIFLUOROETHYLENE AND/OR 1,1,2-TRIFLUOROETHANE

(71) Applicant: ARKEMA FRANCE, COLOMBES (FR)

(72) Inventors: Abdelatif BABA-AHMED, PIERRE-BENITE CEDEX (FR); Alexandre CAMBRODON, Pierre-Benite Cedex (FR); Dominique GARRAIT, Pierre-Benite Cedex (FR); Thierry LANNUZEL, Pierre-Benite Cedex (FR); Cédric LAVY, Pierre-Benite Cedex (FR); Laurent WENDLINGER, Pierre-Benite Cedex (FR); Kevin HISLER, Pierre-Benite Cedex (FR)

(21) Appl. No.: 18/564,245

(22) PCT Filed: Jun. 3, 2022
(86) PCT No.: PCT/FR2022/051053

§ 371 Date: Nov. 27, 2023

(30) Foreign Application Priority Data
Jun. 7, 2021 (FR) FR2105961

(51) Int. Cl. C07C 21/18

(52) U.S. Cl. CPC C07C 21/18

(57) ABSTRACT

The present invention relates to an azeotropic or quasi-azeotropic composition comprising chlorotrifluoroethylene and at least one of the compounds selected from the group consisting of 1,1,2-trifluoroethane, 1,1-difluoroethane and a mixture of the two. The present invention also relates to an azeotropic or quasi-azeotropic composition comprising 1,1,2-trifluoroethane and at least one of the compounds selected from the group consisting of 2-chloro-1,1,1-trifluoroethane, 1-chloro-1,1,2-trifluoroethane and a mixture of the two.

### TECHNICAL FIELD OF THE INVENTION

The present invention relates to azeotropic or quasi-azeotropic compositions comprising chlorotrifluoroethylene and/or 1,1,2-trifluoroethane. These compositions can originate from intermediate compositions during the production of trifluoroethylene, and more particularly originate from the recycling streams employed during the production of trifluoroethylene.

### Technological Background of the Invention

Fluorinated olefins, such as VF3, are known and are used as monomers or comonomers for the manufacture of fluorocarbon polymers exhibiting noteworthy characteristics, in particular excellent chemical resistance and good heat resistance.

A known route for the preparation of trifluoroethylene uses, as starting materials, chlorotrifluoroethylene (CTFE) and hydrogen in the presence of a catalyst and in the gas phase. There is known, in particular from WO 2013/128102, a process for the production of trifluoroethylene by hydrogenolysis of CTFE in the gas phase and in the presence of a catalyst based on a metal from group VIII at atmospheric pressure and at relatively low temperatures. The production of trifluoroethylene is accompanied by a multitude of by-products having, for some, a boiling point close to trifluoroethylene or close to chlorotrifluoroethylene. This involves the implementation of complex purification stages which can result in the consequent loss in the product of interest. In addition, some of these by-products can form azeotropic compositions which render the separation difficult, indeed even impossible.

### SUMMARY OF THE INVENTION

The present invention relates to azeotropic or quasi-azeotropic compositions surprisingly identified.

In particular, according to a first aspect, the present invention relates to an azeotropic or quasi-azeotropic composition comprising chlorotrifluoroethylene and at least one of the compounds selected from the group consisting of 1,1,2-trifluoroethane, 1,1-difluoroethane and a mixture of the two, characterized in that it contains at least 50% by weight of chlorotrifluoroethylene, based on the total weight of said composition.

According to a preferred embodiment, said composition has a boiling point of between −40° C. and 40° C. at a pressure of between 0.5 bara and 8 bara.

According to a preferred embodiment, said composition also comprises at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene and 1,1,2,2-tetrafluoroethane.

According to a preferred embodiment, said composition contains at most 20% by weight of 1,1,2-trifluoroethane, based on the total weight of said composition.

According to a preferred embodiment, said composition contains at most 50% by weight of 1,1-difluoroethane, based on the total weight of said composition.

According to a second aspect, the present invention relates to an azeotropic or quasi-azeotropic composition comprising 1,1,2-trifluoroethane and at least one of the compounds selected from the group consisting of 2-chloro-1,1,1-trifluoroethane, 1-chloro-1,1,2-trifluoroethane and a mixture of the two.

According to a preferred embodiment, said composition has a boiling point of between −10° C. and 70° C. at a pressure of between 0.5 bara and 8 bara.

According to a preferred embodiment, said composition contains between 35% and 99.99% by weight of 1,1,2-trifluoroethane, based on the total weight of said composition.

According to a preferred embodiment, said composition contains at most 65% by weight of 2-chloro-1,1,1-trifluoroethane or at most 38% by weight of 1-chloro-1,1,2-trifluoroethane, based on the total weight of said composition.

### BRIEF DESCRIPTION OF THE FIGURES

FIG. 1 represents the change in the bubble point and in the dew point as a function of the content by weight of CTFE for the CTFE/143 binary mixture at a given pressure.

FIG. 2 represents the change in the bubble point and in the dew point as a function of the content by weight of CTFE for the CTFE/152a binary mixture at a given pressure.

FIG. 3 represents the change in the bubble point and in the dew point as a function of the content by weight of 143 for the 143/133b binary mixture at a given pressure.

FIG. 4 represents the change in the bubble point and in the dew point as a function of the content by weight of 143 for the 143/133a binary mixture at a given pressure.

### DETAILED DESCRIPTION OF THE INVENTION

The present invention relates to azeotropic or quasi-azeotropic compositions.

The expression “quasi-azeotropic” has a broad meaning and is intended to include compositions which are strictly azeotropic and those which behave like an azeotropic mixture.

The volatility of a compound A is represented by the ratio of the molar fraction in the gas phase (y_{A}) to the molar fraction in the liquid phase (x_{A}) under equilibrium conditions (at pressure and temperature equilibrium): α=y_{A}/x_{A}. The volatility of a compound B is represented by the ratio of the molar fraction in the gas phase (y_{B}) to the molar fraction in the liquid phase (x_{B}) under equilibrium conditions (at pressure and temperature equilibrium): α=y_{B}/x_{B}. The relative volatility makes it possible to measure the ease of separation of two compounds A and B. It is the ratio of the volatilities of the two compounds: α_{A,B}=y_{A}x_{B}/x_{A}y_{B}. The greater the volatility, the more the mixture can be easily separated.

In the present invention, a composition is regarded as being azeotropic when the relative volatility between two compounds is equal to 1 or is between 0.99 and 1.01. In the present invention, a composition is regarded as being quasi-azeotropic when the relative volatility between two compounds is between 0.9 and 1.1.

There has surprisingly been discovered the formation of azeotropic or quasi-azeotropic compositions during the process for the production of trifluoroethylene. The presence of these compositions has an impact on the overall process, particularly during the purification and recycling stages.

### A) Composition Comprising Chlorotrifluoroethylene (CTFE)

According to a first aspect, the present invention provides an azeotropic or quasi-azeotropic composition comprising chlorotrifluoroethylene. Preferably, said azeotropic or quasi-azeotropic composition comprises at least one of the compounds selected from the group consisting of 1,1,2-trifluoroethane (143), 1,1-difluoroethane (152a) and a mixture of the two.

The azeotropic or quasi-azeotropic composition can also comprise at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene and 1,1,2,2-tetrafluoroethane.

Thus, said composition can comprise:
a) chlorotrifluoroethylene;
b) at least one of the compounds selected from the group consisting of 1,1,2-trifluoroethane, 1,1-difluoroethane and a mixture of the two; and
c) optionally, at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene and 1,1,2,2-tetrafluoroethane.

According to a preferred embodiment, said azeotropic or quasi-azeotopic composition can comprise chlorotrifluoroethylene, 1,1,2-trifluoroethane and at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene and 1,1,2,2-tetrafluoroethane.

Alternatively, said azeotropic or quasi-azeotropic composition can comprise chlorotrifluoroethylene, 1,1-difluoroethane and at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene and 1,1,2,2-tetrafluoroethane.

Alternatively, said azeotropic or quasi-azeotropic composition can comprise chlorotrifluoroethylene, 1,1,2-trifluoroethane, 1,1-difluoroethane and at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene and 1,1,2,2-tetrafluoroethane.

Preferably, said composition has a boiling point of between −40° C. and 40° C., more preferentially between −35° C. and 25° C. In particular, said composition has a boiling point of between −40° ° C. and 40° C. at a pressure of between 0.5 bara and 8 bara. More particularly, said composition has a boiling point of between −35° C. and 25° C. at a pressure of from 1 bara to 6 bara.

According to a preferred embodiment, said composition comprises at least 50% by weight of chlorotrifluoroethylene, based on the total weight of said composition. Preferably, said composition comprises at least 52% by weight, or at least 54% by weight, or at least 56% by weight, or at least 58% by weight, or at least 60% by weight, or at least 62% by weight, or at least 64% by weight, or at least 66% by weight, or at least 68% by weight, or at least 70% by weight, or at least 72% by weight, or at least 74% by weight, or at least 76% by weight, or at least 78% by weight, or at least 80% by weight, or at least 82% by weight, or at least 84% by weight, or at least 86% by weight, or at least 88% by weight, or at least 90% by weight, or at least 92% by weight, or at least 94% by weight, or at least 96% by weight, or at least 98% by weight, or at least 99.99% by weight of chlorotrifluoroethylene, based on the total weight of said composition.

According to a preferred embodiment, said composition comprises at most 20% by weight of 1,1,2-trifluoroethane, based on the total weight of said composition. Preferably, said composition comprises at most 18% by weight, or at most 16% by weight, or at most 14% by weight, or at most 12% by weight, or at most 10% by weight, or at most 8% by weight, or at most 6% by weight, or at most 4% by weight, or at most 2% by weight, or at most 0.01% by weight of 1,1,2-trifluoroethane, based on the total weight of said composition.

According to a preferred embodiment, said composition comprises at most 50% by weight of 1,1-difluoroethane, based on the total weight of said composition. Preferably, said composition comprises at most 48% by weight, or at most 46% by weight, or at most 44% by weight, or at most 42% by weight, or at most 40% by weight, or at most 38% by weight, or at most 36% by weight, or at most 34% by weight, or at most 32% by weight, or at most 30% by weight, or at most 28% by weight, or at most 26% by weight of 1,1-difluoroethane, based on the total weight of said composition.

As mentioned above, said composition according to the present invention can comprise at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene and 1,1,2,2-tetrafluoroethane. Thus, said composition can comprise at least two, or at least three, or at least four, or at least five, or at least six, or at least seven, or at least eight, or at least nine, or at least ten, or all of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene and 1,1,2,2-tetrafluoroethane.

According to a preferred embodiment, said composition contains less than 10% by weight of said additional compounds, based on the total weight of said composition. Preferably, said composition contains less than 8% by weight, or less than 6% by weight, or less than 4% by weight, or less than 2% by weight, or less than 1% by weight, or less than 0.5% by weight, or less than 0.1% by weight, or less than 0.05% by weight, or less than 0.01% by weight of said additional compounds, based on the total weight of said composition.

### A-i) Composition Based on CTFE and 143

According to a particular embodiment of the first aspect of the present invention, said azeotropic or quasi-azeotropic composition comprises chlorotrifluoroethylene and 1,1,2-trifluoroethane.

Advantageously, said composition comprises from 80% to 99.99% by weight of chlorotrifluoroethylene, based on the total weight of said composition. Preferably, said composition comprises from 85% to 99.99% by weight of chlorotrifluoroethylene, based on the total weight of said composition. In particular, said composition comprises from 90% to 99.99% by weight of chlorotrifluoroethylene, based on the total weight of said composition.

Advantageously, said composition comprises from 0.01% to 20% by weight of 1,1,2-trifluoroethane, based on the total weight of said composition. Preferably, said composition comprises from 0.01% to 15% by weight of 1,1,2-trifluoroethane, based on the total weight of said composition. In particular, said composition comprises from 0.01% to 10% by weight of 1,1,2-trifluoroethane, based on the total weight of said composition.

Preferably, said composition has a boiling point of between −40° C. and 40° C., more preferentially between −35° C. and 25° C. In particular, said composition has a boiling point of between −40° C. and 40° C. at a pressure of between 0.5 bara and 8 bara. More particularly, said composition has a boiling point of between −35° C. and 25° C. at a pressure of from 1 bara to 6 bara.

Thus, said azeotropic or quasi-azeotropic composition can comprise from 80% to 99.99% by weight of chlorotrifluoroethylene and from 0.01% to 20% by weight of 1,1,2-trifluoroethane, based on the total weight of said composition, and has a boiling point of between −40° C. and 40° C. at a pressure of between 0.5 bara and 8 bara. Advantageously, said azeotropic or quasi-azeotropic composition comprises from 85% to 99.99% by weight of chlorotrifluoroethylene and from 0.01% to 15% by weight of 1,1,2-trifluoroethane, based on the total weight of said composition; and has a boiling point of between −40° C. and 40° C. at a pressure of between 0.5 bara and 8 bara. More particularly, said azeotropic or quasi-azeotropic composition comprises from 90% to 99.99% by weight of chlorotrifluoroethylene and from 0.01% to 10% by weight of 1,1,2-trifluoroethane, based on the total weight of said composition, and has a boiling point of between −30° C. and 25° C. at a pressure of from 1 bara to 6 bara.

Preferably, said azeotropic or quasi-azeotropic composition comprises, preferably consists of:
from 90% to 99.99% by weight of chlorotrifluoroethylene and from 0.01% to 10% by weight of 1,1,2-trifluoroethane, based on the total weight of said composition, and has a boiling point of between 20° C. and 25° C. at a pressure of 6 bara;
from 92% to 99.99% by weight of chlorotrifluoroethylene and from 0.01% to 8% by weight of 1,1,2-trifluoroethane, based on the total weight of said composition, and has a boiling point of between 12° C. and 17° C. at a pressure of 5 bara;
from 92% to 99.99% by weight of chlorotrifluoroethylene and from 0.01% to 8% by weight of 1,1,2-trifluoroethane, based on the total weight of said composition, and has a boiling point of between 5° C. and 10° C. at a pressure of 4 bara;
from 94% to 99.99% by weight of chlorotrifluoroethylene and from 0.01% to 6% by weight of 1,1,2-trifluoroethane, based on the total weight of said composition, and has a boiling point of between −3° C. and 2° C. at a pressure of 3 bara;
from 96% to 99.99% by weight of chlorotrifluoroethylene and from 0.01% to 4% by weight of 1,1,2-trifluoroethane, based on the total weight of said composition, and has a boiling point of between −15° C. and −10° C. at a pressure of 2 bara;
from 98% to 99.99% by weight of chlorotrifluoroethylene and from 0.01% to 2% by weight of 1,1,2-trifluoroethane, based on the total weight of said composition, and has a boiling point of between −30° C. and −25° C. at a pressure of 1.013 bara;
from 98% to 99.99% by weight of chlorotrifluoroethylene and from 0.01% to 2% by weight of 1,1,2-trifluoroethane, based on the total weight of said composition, and has a boiling point of between −30° C. and −25° C. at a pressure of 1 bara.

In an advantageously preferred way, said composition can also comprise 1,1-difluoroethane or additional compounds as defined above in the present patent application. When it contains it/them, said composition can comprise one, two, three, four, five, six, seven, eight, nine, ten, eleven or all of said additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane.

Thus, said azeotropic or quasi-azeotropic composition comprises, preferably consists of:
from 90% to 99.99% by weight of chlorotrifluoroethylene, from 0.01% to 10% by weight of 1,1,2-trifluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane, based on the total weight of said composition, said composition having a boiling point of between 20° C. and 25° C. at a pressure of 6 bara;
from 92% to 99.99% by weight of chlorotrifluoroethylene, from 0.01% to 8% by weight of 1,1,2-trifluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane, based on the total weight of said composition, said composition having a boiling point of between 12° C. and 17° C. at a pressure of 5 bara;
from 92% to 99.99% by weight of chlorotrifluoroethylene, from 0.01% to 8% by weight of 1,1,2-trifluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane, based on the total weight of said composition, said composition having a boiling point of between 5° C. and 10° C. at a pressure of 4 bara;
from 94% to 99.99% by weight of chlorotrifluoroethylene, from 0.01% to 6% by weight of 1,1,2-trifluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane, based on the total weight of said composition, said composition having a boiling point of between −3° C. and 2° C. at a pressure of 3 bara;
from 96% to 99.99% by weight of chlorotrifluoroethylene, from 0.01% to 4% by weight of 1,1,2-trifluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane, based on the total weight of said composition, said composition having a boiling point of between −15° C. and −10° C. at a pressure of 2 bara;
from 98% to 99.99% by weight of chlorotrifluoroethylene, from 0.01% to 2% by weight of 1,1,2-trifluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane, based on the total weight of said composition, said composition having a boiling point of between −30° C. and −25° C. at a pressure of 1.013 bara;
from 98% to 99.99% by weight of chlorotrifluoroethylene, from 0.01% to 2% by weight of 1,1,2-trifluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane, based on the total weight of said composition, said composition having a boiling point of between −30° C. and −25° C. at a pressure of 1 bara.

More preferably, the present invention provides an azeotropic composition comprising, preferably consisting of, from 92% to 93% by weight of chlorotrifluoroethylene and from 7% to 8% by weight of 1,1,2-trifluoroethane, said composition having a boiling point of 21° C. to 22° C. at a pressure of 6 bara. Preferably, said azeotropic composition comprises, more preferentially consists of, from 92% to 93% by weight of chlorotrifluoroethylene and from 7% to 8% by weight of 1,1,2-trifluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane, based on the total weight of said composition, said composition having a boiling point of 21° C. to 22° C. at a pressure of 6 bara. In particular, said azeotropic composition consists of 92.5% by weight of chlorotrifluoroethylene and 7.5% by weight of 1,1,2-trifluoroethane, said composition having a boiling point of 21.3° C. at a pressure of 6 bara.

More preferably, the present invention provides an azeotropic composition comprising, preferably consisting of, from 93% to 94% by weight of chlorotrifluoroethylene and from 6% to 7% by weight of 1,1,2-trifluoroethane, said composition having a boiling point of 15° C. to 16° C. at a pressure of 5 bara. Preferably, said azeotropic composition comprises, more preferentially consists of, from 93% to 94% by weight of chlorotrifluoroethylene and from 6% to 7% by weight of 1,1,2-trifluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane, based on the total weight of said composition, said composition having a boiling point of 15° C. to 16° C. at a pressure of 5 bara. In particular, said azeotropic composition consists of 93.4% by weight of chlorotrifluoroethylene and 6.6% by weight of 1,1,2-trifluoroethane, said composition having a boiling point of 15.2° C. at a pressure of 5 bara.

More preferably, the present invention provides an azeotropic composition comprising, preferably consisting of, from 94% to 95% by weight of chlorotrifluoroethylene and from 5% to 6% by weight of 1,1,2-trifluoroethane, said composition having a boiling point of 8° C. to 9° C. at a pressure of 4 bara. Preferably, said azeotropic composition comprises, more preferentially consists of, from 94% to 95% by weight of chlorotrifluoroethylene and from 5% to 6% by weight of 1,1,2-trifluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane, based on the total weight of said composition, said composition having a boiling point of 8° C. to 9° C. at a pressure of 4 bara. In particular, said azeotropic composition consists of 94.4% by weight of chlorotrifluoroethylene and 5.6% by weight of 1,1,2-trifluoroethane, said composition having a boiling point of 8.1° C. at a pressure of 4 bara.

More preferably, the present invention provides an azeotropic composition comprising, preferably consisting of, from 95% to 96% by weight of chlorotrifluoroethylene and from 4% to 5% by weight of 1,1,2-trifluoroethane, said composition having a boiling point of −1° C. to 0° C. at a pressure of 3 bara. Preferably, said azeotropic composition comprises, more preferentially consists of, from 95% to 96% by weight of chlorotrifluoroethylene and from 4% to 5% by weight of 1,1,2-trifluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane, based on the total weight of said composition, said composition having a boiling point of −1° C. to 0° C. at a pressure of 3 bara. In particular, said azeotropic composition consists of 95.7% by weight of chlorotrifluoroethylene and 4.3% by weight of 1,1,2-trifluoroethane, said composition having a boiling point of −0.5° C. at a pressure of 3 bara.

More preferably, the present invention provides an azeotropic composition comprising, preferably consisting of, from 97% to 98% by weight of chlorotrifluoroethylene and from 2% to 3% by weight of 1,1,2-trifluoroethane, said composition having a boiling point of −12° C. to −11° C. at a pressure of 2 bara. Preferably, said azeotropic composition comprises, more preferentially consists of, from 97% to 98% by weight of chlorotrifluoroethylene and from 3% to 4% by weight of 1,1,2-trifluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane, based on the total weight of said composition, said composition having a boiling point of −12° C. to −11ºC at a pressure of 2 bara. In particular, said azeotropic composition consists of 97.4% by weight of chlorotrifluoroethylene and 2.6% by weight of 1,1,2-trifluoroethane, said composition having a boiling point of −11.7° C. at a pressure of 2 bara.

More preferably, the present invention provides an azeotropic composition comprising, preferably consisting of, from 99.5% to 99.99% by weight of chlorotrifluoroethylene and from 0.01% to 0.5% by weight of 1,1,2-trifluoroethane, said composition having a boiling point of −28° C. to −29° C. at a pressure of 1.013 bara. Preferably, said azeotropic composition comprises, more preferentially consists of, from 99.5% to 99.9% by weight of chlorotrifluoroethylene and from 0.01% to 0.5% by weight of 1,1,2-trifluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1-difluoroethane, based on the total weight of said composition, said composition having a boiling point of −28° C. to −29° C. at a pressure of 1.013 bara. In particular, said azeotropic composition consists of 99.98% by weight of chlorotrifluoroethylene and 0.02% by weight of 1,1,2-trifluoroethane, said composition having a boiling point of −28.4° C. at a pressure of 1.013 bara.

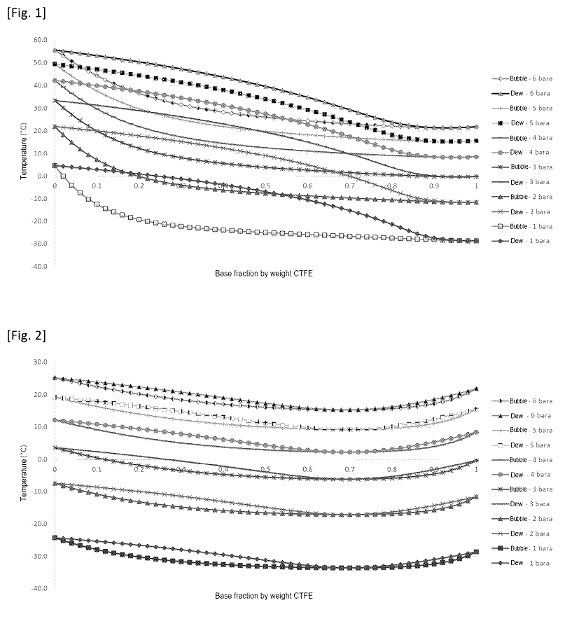

FIG. 1 represents the change in the bubble point and in the dew point as a function of the content by weight of chlorotrifluoroethylene for the chlorotrifluoroethylene/1,1,2-trifluoroethane binary mixture at a pressure of 1 to 6 bara.

### A-ii) Composition Based on CTFE and 152a

According to another particular embodiment of the first aspect of the present invention, said azeotropic or quasi-azeotropic composition comprises chlorotrifluoroethylene and 1,1-difluoroethane.

Advantageously, said azeotropic or quasi-azeotropic composition comprises from 60% to 80% by weight of chlorotrifluoroethylene, based on the total weight of said composition. Preferably, said azeotropic or quasi-azeotropic composition comprises from 63% to 77% by weight of chlorotrifluoroethylene, based on the total weight of said composition. In particular, said azeotropic or quasi-azeotropic composition comprises from 66% to 74% by weight of chlorotrifluoroethylene, based on the total weight of said composition.

Advantageously, said azeotropic or quasi-azeotropic composition comprises from 20% to 40% by weight of 1,1-difluoroethane, based on the total weight of said composition. Preferably, said azeotropic or quasi-azeotropic composition comprises from 23% to 37% by weight of 1,1-difluoroethane, based on the total weight of said composition. In particular, said azeotropic or quasi-azeotropic composition comprises from 26% to 34% by weight of 1,1-difluoroethane, based on the total weight of said composition.

Preferably, said composition has a boiling point of between −40° C. and 30° C., more preferentially between −35° C. and 16° C. In particular, said composition has a boiling point of between −40° C. and 30° C. at a pressure of between 0.5 bara and 8 bara. More particularly, said composition has a boiling point of between −35° C. and 16° C. at a pressure of from 1 bara to 6 bara.

Thus, said azeotropic or quasi-azeotropic composition can comprise from 60% to 80% by weight of chlorotrifluoroethylene and from 20% to 40% by weight of 1,1-difluoroethane, based on the total weight of said composition, and has a boiling point of between −40° C. and 30° C. at a pressure of from 0.5 bara to 8 bara. Advantageously, said azeotropic or quasi-azeotropic composition can comprise from 63% to 77% by weight of chlorotrifluoroethylene and from 23% to 37% by weight of 1,1-difluoroethane, based on the total weight of said composition, and has a boiling point of between −40° C. and 30° C. at a pressure of from 0.5 bara to 8 bara. More particularly, said azeotropic or quasi-azeotropic composition comprises from 66% to 74% by weight of chlorotrifluoroethylene and from 26% to 34% by weight of 1,1-difluoroethane, based on the total weight of said composition, and has a boiling point of between −35° C. and 16° C. at a pressure of from 1 bara to 6 bara.

Preferably, said azeotropic or quasi-azeotropic composition comprises, preferably consists of:
from 66% to 74% by weight of chlorotrifluoroethylene and from 26% to 34% by weight of 1,1-difluoroethane, based on the total weight of said composition, and has a boiling point of between 14° C. and 17° C. at a pressure of 6 bara;
from 66% to 74% by weight of chlorotrifluoroethylene and from 26% to 34% by weight of 1,1-difluoroethane and has a boiling point of between 7ºC and 11° C. at a pressure of 5 bara;
from 66% to 74% by weight of chlorotrifluoroethylene and from 26% to 34% by weight of 1,1-difluoroethane and has a boiling point of between 0° C. and 4° C. at a pressure of 4 bara;
from 66% to 74% by weight of chlorotrifluoroethylene and from 26% to 34% by weight of 1,1-difluoroethane and has a boiling point of between −8° C. and −4° C. at a pressure of 3 bara;
from 66% to 74% by weight of chlorotrifluoroethylene and from 26% to 34% by weight of 1,1-difluoroethane and has a boiling point of between −15° C. and −19° C. at a pressure of 2 bara;
from 68% to 74% by weight of chlorotrifluoroethylene and from 26% to 32% by weight of 1,1-difluoroethane and has a boiling point of between −32° C. and −36° C. at a pressure of 1 bara.

In an advantageously preferred way, said composition can also comprise 1,1,2-trifluoroethane or additional compounds as defined above in the present patent application. When it contains it/them, said composition can comprise one, two, three, four, five, six, seven, eight, nine, ten, eleven or all of said additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1,2-trifluoroethane.

Thus, said azeotropic or quasi-azeotropic composition comprises, preferably consists of:
from 66% to 74% by weight of chlorotrifluoroethylene, from 26% to 34% by weight of 1,1-difluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1,2-trifluoroethane, based on the total weight of said composition, said composition having a boiling point of between 14° C. and 17° C. at a pressure of 6 bara;
from 66% to 74% by weight of chlorotrifluoroethylene, from 26% to 34% by weight of 1,1-difluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1,2-trifluoroethane, based on the total weight of said composition, said composition having a boiling point of between 7° C. and 11° C. at a pressure of 5 bara;
from 66% to 74% by weight of chlorotrifluoroethylene, from 26% to 34% by weight of 1,1-difluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1,2-trifluoroethane, based on the total weight of said composition, said composition having a boiling point of between 0° C. and 4° C. at a pressure of 4 bara;
from 66% to 74% by weight of chlorotrifluoroethylene, from 26% to 34% by weight of 1,1-difluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1,2-trifluoroethane, based on the total weight of said composition, said composition having a boiling point of between −8° C. and −4° C. at a pressure of 3 bara;
from 66% to 74% by weight of chlorotrifluoroethylene, from 26% to 34% by weight of 1,1-difluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1,2-trifluoroethane, based on the total weight of said composition, said composition having a boiling point of between −15° C. and −19° C. at a pressure of 2 bara;
from 68% to 74% by weight of chlorotrifluoroethylene, from 26% to 32% by weight of 1,1-difluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1,2-trifluoroethane, based on the total weight of said composition, said composition having a boiling point of between −32° C. and −36° C. at a pressure of 1 bara.

More preferably, the present invention provides an azeotropic composition comprising, preferably consisting of, from 69% to 70% by weight of chlorotrifluoroethylene and from 30% to 31% by weight of 1,1-difluoroethane, based on the total weight of said composition, said composition having a boiling point of −35° C. to 16° C. at a pressure of 1 to 6 bara. In a preferentially preferred way, the present invention provides an azeotropic composition comprising, preferably consisting of, from 69% to 70% by weight of chlorotrifluoroethylene, from 30% to 31% by weight of 1,1-difluoroethane, and optionally the remainder as at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene, 1,1,2,2-tetrafluoroethane and 1,1,2-trifluoroethane, based on the total weight of said composition, said composition having a boiling point of −35° C. to 16° C. at a pressure of 1 to 6 bara.

In particular, said azeotropic composition consists of 69.1% by weight of chlorotrifluoroethylene and 30.9% by weight of 1,1-difluoroethane, based on the total weight of the composition, and has a boiling point of 15.3° C. at 6 bara.

In particular, said azeotropic composition consists of 69.2% by weight of chlorotrifluoroethylene and 30.8% by weight of 1,1-difluoroethane, based on the total weight of the composition, and has a boiling point of 9.2° C. at 5 bara.

In particular, said azeotropic composition consists of 69.3% by weight of chlorotrifluoroethylene and 30.7% by weight of 1,1-difluoroethane, based on the total weight of the composition, and has a boiling point of 2.2° C. at 4 bara.

In particular, said azeotropic composition consists of 69.4% by weight of chlorotrifluoroethylene and 30.6% by weight of 1,1-difluoroethane, based on the total weight of the composition, and has a boiling point of −6.2° C. at 3 bara.

In particular, said azeotropic composition consists of 69.5% by weight of chlorotrifluoroethylene and 30.5% by weight of 1,1-difluoroethane, based on the total weight of the composition, and has a boiling point of −17.2° C. at 2 bara.

In particular, said azeotropic composition consists of 69.7% by weight of chlorotrifluoroethylene and 30.3% by weight of 1,1-difluoroethane, based on the total weight of the composition, and has a boiling point of −33.7° C. at 1 bara.

FIG. 2 represents the change in the bubble point and in the dew point as a function of the content by weight of chlorotrifluoroethylene for the chlorotrifluoroethylene/1,1-difluoroethane binary mixture at a pressure of 1 to 6 bara.

The presence of azeotropic or quasi-azeotropic compositions as defined in the present invention is confirmed by the data resulting from [FIG. 1] and [FIG. 2]. The relative volatilities of each of the compositions presented in FIGS. 1 and 2 were calculated at the liquid-vapor equilibrium.

### B) Composition Comprising 1,1,2-Trifluoroethane (143)

According to a second aspect, the present invention relates to an azeotropic or quasi-azeotropic composition comprising 1,1,2-trifluoroethane. Preferably, said composition comprises at least one of the compounds selected from the group consisting of 2-chloro-1,1,1-trifluoroethane (133a), 1-chloro-1,1,2-trifluoroethane (133b) and a mixture of the two.

According to a particular embodiment, said composition can also comprise at least one of the additional compounds selected from the group consisting of 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane.

Thus, said azeotropic or quasi-azeotropic composition can comprise:
a) 1,1,2-trifluoroethane;
b) at least one of the compounds selected from the group consisting of 2-chloro-1,1,1-trifluoroethane (133a), 1-chloro-1,1,2-trifluoroethane (133b) and a mixture of the two; and
c) optionally at least one of the additional compounds selected from the group consisting of 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane.

According to a preferred embodiment, said azeotropic or quasi-azeotropic composition can comprise 1,1,2-trifluoroethane, 2-chloro-1,1,1-trifluoroethane and optionally at least one of the additional compounds selected from the group consisting of 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane.

Alternatively, said azeotropic or quasi-azeotropic composition can comprise 1,1,2-trifluoroethane, 1-chloro-1,1,2-trifluoroethane and optionally at least one of the additional compounds selected from the group consisting of 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane.

Alternatively, said azeotropic or quasi-azeotropic composition can comprise 1,1,2-trifluoroethane, 2-chloro-1,1,1-trifluoroethane, 1-chloro-1,1,2-trifluoroethane and optionally at least one of the additional compounds selected from the group consisting of 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane.

Preferably, said azeotropic or quasi-azeotropic composition has a boiling point of between −10° C. and 70° C. In particular, said azeotropic or quasi-azeotropic composition has a boiling point of between −10° C. and 70° C. at a pressure of between 0.5 bara and 8 bara. More particularly, said azeotropic or quasi-azeotropic composition has a boiling point of between 0° C. and 65° C. at a pressure of 1 to 6 bara.

According to a particular embodiment, said composition comprises at least 30% by weight, advantageously at least 32% by weight, preferably at least 34% by weight, in particular at least 36% by weight, more particularly at least 38% by weight, of 1,1,2-trifluoroethane, based on the total weight of said composition.

According to a particular embodiment, said composition comprises at most 46% by weight, advantageously at most 44% by weight, preferably at most 42% by weight, in particular at most 40% by weight, more particularly at most 38% by weight, of 1-chloro-1,1,2-trifluoroethane, based on the total weight of said composition.

According to a particular embodiment, said composition comprises at most 66% by weight, advantageously at most 65% by weight, preferably at most 64% by weight, in particular at most 63% by weight, more particularly at most 62% by weight, of 2-chloro-1,1,1-trifluoroethane, based on the total weight of said composition.

As mentioned above, said composition according to the present invention can comprise at least one of the additional compounds selected from the group consisting of 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane. Thus, said composition can comprise at least two, or at least three, or at least four, or at least five, or at least six, or at least seven, or at least eight, or at least nine, or at least ten, or at least eleven, or all of the additional compounds selected from the group consisting of 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane.

According to a preferred embodiment, said composition contains less than 10% by weight of said additional compounds, based on the total weight of the composition. Preferably, said composition less than 8% by weight, or less than 6% by weight, or less than 4% by weight, or less than 2% by weight, or less than 1% by weight, or less than 0.5% by weight, or less than 0.1% by weight, or less than 0.05% by weight, or less than 0.01% by weight of said additional compounds, based on the total weight of the composition.

### B-i) Compositions Based on 143 and 133b

According to a particular embodiment of the second aspect of the present invention, said azeotropic or quasi-azeotropic composition comprises 1,1,2-trifluoroethane and 1-chloro-1,1,2-trifluoroethane.

Advantageously, said composition comprises from 55% to 99.99% by weight of 1,1,2-trifluoroethane, based on the total weight of said composition. Preferably, said composition comprises from 60% to 99.99% by weight of 1,1,2-trifluoroethane, based on the total weight of said composition. In particular, said composition comprises from 62% to 99.99% by weight of 1,1,2-trifluoroethane, based on the total weight of said composition.

Advantageously, said composition comprises from 0.01% to 45% by weight of 1-chloro-1,1,2-trifluoroethane, based on the total weight of said composition. Preferably, said composition comprises from 0.01% to 40% by weight of 1-chloro-1,1,2-trifluoroethane, based on the total weight of said composition. In particular, said composition comprises from 0.01% to 38% by weight of 1-chloro-1,1,2-trifluoroethane, based on the total weight of said composition.

Preferably, said composition has a boiling point of between 0° C. and 40° C., more preferentially between 0° C. and 35° C. In particular, said composition has a boiling point of between 0° C. and 40° C. at a pressure of 1 bara to 3 bara. More particularly, said composition has a boiling point of between 0° C. and 35° C. at a pressure of from 1 bara to 3 bara.

More preferentially, said azeotropic or quasi-azeotropic composition comprises from 60% to 99.99% by weight of 1,1,2-trifluoroethane and from 0.01% to 40% by weight of 1-chloro-1,1,2-trifluoroethane, based on the total weight of said composition, and has a boiling point of between 0° C. and 40° ° C. at a pressure of 1 to 3 bara.

In particular, said azeotropic or quasi-azeotropic composition comprises from 62% to 99.99% by weight of 1,1,2-trifluoroethane and from 0.01% to 38% by weight of 1-chloro-1,1,2-trifluoroethane, based on the total weight of said composition, and has a boiling point of between 0° C. and 35° C. at a pressure of 1 to 3 bara.

Preferably, said azeotropic or quasi-azeotropic composition comprises, preferably consists of:
from 62% to 84% by weight of 1,1,2-trifluoroethane and from 16% to 38% by weight of 1-chloro-1,1,2-trifluoroethane, based on the total weight of said composition, and has a boiling point of 2° C. to 6° ° C. at a pressure of 1 bara;
from 62% to 84% by weight of 1,1,2-trifluoroethane and from 16% to 38% by weight of 1-chloro-1,1,2-trifluoroethane, based on the total weight of said composition, and has a boiling point of 2° C. to 6° C. at a pressure of 1.013 bara;
from 74% to 99.99% by weight of 1,1,2-trifluoroethane and from 0.01% to 26% by weight of 1-chloro-1,1,2-trifluoroethane, based on the total weight of said composition, and has a boiling point of 20° C. to 24° C. at a pressure of 2 bara;
from 88% to 99.99% by weight of 1,1,2-trifluoroethane and from 0.01% to 12% by weight of 1-chloro-1,1,2-trifluoroethane, based on the total weight of said composition, and has a boiling point of 31° C. to 35° C. at a pressure of 3 bara.

In an advantageously preferred way, said composition can also comprise 2-chloro-1,1,1-trifluoroethane or additional compounds as defined above according to the second aspect of the present invention. When it contains it/them, said composition can comprise one, two, three, four, five, six, seven, eight, nine, ten, eleven or all of said additional compounds selected from the group consisting of 2-chloro-1,1,1-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane.

Said azeotropic or quasi-azeotropic composition can also comprise, preferably consist of:
from 62% to 84% by weight of 1,1,2-trifluoroethane, from 16% to 38% by weight of 1-chloro-1,1,2-trifluoroethane and optionally the remainder as at least one of the additional compounds selected from the group consisting of 2-chloro-1,1,1-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, the latter having a boiling point of 2° C. to 6° C. at a pressure of 1 bara;
from 62% to 84% by weight of 1,1,2-trifluoroethane, from 16% to 38% by weight of 1-chloro-1,1,2-trifluoroethane and optionally the remainder as at least one of the additional compounds selected from the group consisting of 2-chloro-1,1,1-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, the latter having a boiling point of 2° C. to 6° C. at a pressure of 1.013 bara;
from 74% to 99.99% by weight of 1,1,2-trifluoroethane, from 0.01% to 26% by weight of 1-chloro-1,1,2-trifluoroethane and optionally the remainder as at least one of the additional compounds selected from the group consisting of 2-chloro-1,1,1-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, the latter having a boiling point of 20° C. to 24° C. at a pressure of 2 bara;
from 88% to 99.99% by weight of 1,1,2-trifluoroethane, from 0.01% to 12% by weight of 1-chloro-1,1,2-trifluoroethane and optionally the remainder as at least one of the additional compounds selected from the group consisting of 2-chloro-1,1,1-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, the latter having a boiling point of 31° C. to 35° C. at a pressure of 3 bara.

In a particularly preferred way, the present invention provides azeotropic compositions comprising, preferably consisting of, from 71% to 72% by weight of 1,1,2-trifluoroethane and from 28% to 29% by weight of 1-chloro-1,1,2-trifluoroethane, based on the total weight of said composition, and having a boiling point of 3° C. to 4° C. at a pressure of 1 bara or a boiling point of 4ºC to 5° C. at a pressure of 1.013 bara.

In a particularly preferred way, the present invention provides azeotropic compositions comprising, preferably consisting of, from 71% to 72% by weight of 1,1,2-trifluoroethane, from 28% to 29% by weight of 1-chloro-1,1,2-trifluoroethane, and optionally the remainder as at least one of the additional compounds selected from the group consisting of 2-chloro-1,1,1-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, and having a boiling point of 3° C. to 4° C. at a pressure of 1 bara or a boiling point of 4° ° C. to 5° C. at a pressure of 1.013 bara.

More particularly preferably, said azeotropic composition consists of 71.6% of 1,1,2-trifluoroethane and 28.4% by weight of 1-chloro-1,1,2-trifluoroethane, based on the total weight of said composition, and has a boiling point of 3.9° C. at a pressure of 1 bara.

More particularly preferably, said azeotropic composition consists of 71.8% of 1,1,2-trifluoroethane and 28.2% by weight of 1-chloro-1,1,2-trifluoroethane, based on the total weight of said composition, and has a boiling point of 4.2° C. at a pressure of 1.013 bara.

In a particularly preferred way, the present invention also provides azeotropic compositions comprising, preferably consisting of, from 88.5% to 89.5% by weight of 1,1,2-trifluoroethane and from 10.5% to 11.5% by weight of 1-chloro-1,1,2-trifluoroethane, based on the total weight of said composition, and having a boiling point of 21° C. to 23° C. at a pressure of 2 bara.

In a particularly preferred way, the present invention also provides azeotropic compositions comprising, preferably consisting of, from 88.5% to 89.5% by weight of 1,1,2-trifluoroethane, from 10.5% to 11.5% by weight of 1-chloro-1,1,2-trifluoroethane and optionally the remainder as at least one of the additional compounds selected from the group consisting of 2-chloro-1,1,1-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, and having a boiling point of 21° C. to 23° C. at a pressure of 2 bara.

More particularly preferably, said azeotropic composition consists of 88.8% by weight of 1,1,2-trifluoroethane and 10.2% by weight of 1-chloro-1,1,2-trifluoroethane, based on the total weight of said composition, and has a boiling point of 21.8° C. at a pressure of 2 bara.

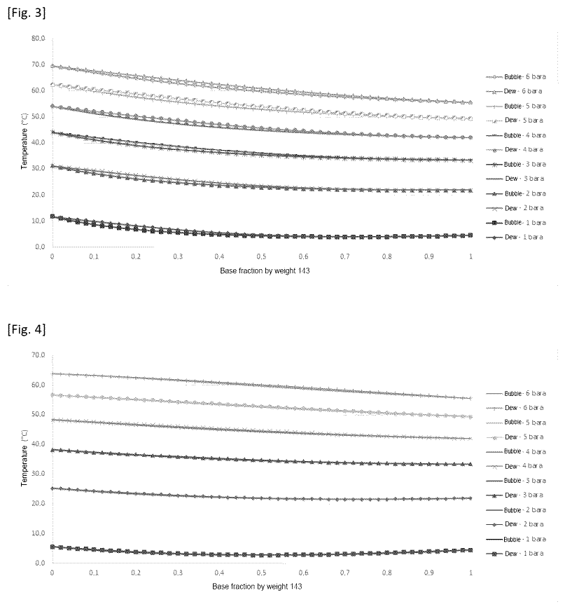

FIG. 3 represents the change in the bubble point and in the dew point as a function of the content by weight of 1,1,2-trifluoroethane for the 1,1,2-trifluoroethane/1-chloro-1,1,2-trifluoroethane binary mixture at a pressure of 1 to 6 bara.

### B-ii) Composition Based on 143 and 133a

According to another particular embodiment of the second aspect of the present invention, said azeotropic or quasi-azeotropic composition comprises 1,1,2-trifluoroethane and 2-chloro-1,1,1-trifluoroethane.

Advantageously, said composition comprises from 30% to 99.99% by weight of 1,1,2-trifluoroethane, based on the total weight of said composition. Preferably, said composition comprises from 35% to 99.99% by weight of 1,1,2-trifluoroethane, based on the total weight of said composition. In particular, said composition comprises from 38% to 99.99% by weight of 1,1,2-trifluoroethane, based on the total weight of said composition.

Advantageously, said composition comprises from 0.01% to 70% by weight of 2-chloro-1,1,1-trifluoroethane, based on the total weight of said composition. Preferably, said composition comprises from 0.01% to 65% by weight of 2-chloro-1,1,1-trifluoroethane, based on the total weight of said composition. In particular, said composition comprises from 0.01% to 62% by weight of 2-chloro-1,1,1-trifluoroethane, based on the total weight of said composition.

Preferably, said composition has a boiling point of between 0° C. and 40° C., more preferentially between 0° C. and 35° C. In particular, said composition has a boiling point of between 0° C. and 40° C. at a pressure of 1 bara to 3 bara. More particularly, said composition has a boiling point of between 0° C. and 35° C. at a pressure of from 1 bara to 3 bara.

According to a preferred embodiment, said azeotropic or quasi-azeotropic composition comprises from 35% to 99.99% by weight of 1,1,2-trifluoroethane and from 0.01% to 65% by weight of 2-chloro-1,1,1-trifluoroethane, based on the total weight of said composition. Preferably, said azeotropic or quasi-azeotropic composition comprises from 35% to 99.99% by weight of 1,1,2-trifluoroethane and from 0.01% to 65% by weight of 2-chloro-1,1,1-trifluoroethane, based on the total weight of said composition, and has a boiling point of between 0° C. and 40° ° C. at a pressure of 1 to 3 bara.

According to a particular embodiment, said azeotropic or quasi-azeotropic composition comprises from 38% to 99.99% by weight of 1,1,2-trifluoroethane and from 0.01% to 62% by weight of 2-chloro-1,1,1-trifluoroethane, based on the total weight of said composition. Preferably, said azeotropic or quasi-azeotropic composition comprises from 38% to 99.99% by weight of 1,1,2-trifluoroethane and from 0.01% to 62% by weight of 2-chloro-1,1,1-trifluoroethane, based on the total weight of said composition, and has a boiling point of between 0° C. and 35° C. at a pressure of 1 to 3 bara.

Preferably, said azeotropic or quasi-azeotropic composition comprises, preferably consists of:
from 38% to 66% by weight of 1,1,2-trifluoroethane and from 34% to 62% by weight of 2-chloro-1,1,1-trifluoroethane, based on the total weight of said composition, having a boiling point of 0° C. to 5° C. at a pressure of 1 bara;
from 38% to 66% by weight of 1,1,2-trifluoroethane and from 34% to 62% by weight of 2-chloro-1,1,1-trifluoroethane, based on the total weight of said composition, having a boiling point of 0° C. to 5° C. at a pressure of 1.013 bara;
from 50% to 94% by weight of 1,1,2-trifluoroethane and from 6% to 50% by weight of 2-chloro-1,1,1-trifluoroethane, based on the total weight of said composition, having a boiling point of 18° C. to 23° C. at a pressure of 2 bara;
from 72% to 99.99% by weight of 1,1,2-trifluoroethane and from 0.01% to 28% by weight of 2-chloro-1,1,1-trifluoroethane, based on the total weight of said composition, having a boiling point of 31° C. to 35° C. at a pressure of 3 bara.

In an advantageously preferred way, said composition can also comprise 1-chloro-1,1,2-trifluoroethane or additional compounds as defined above according to the second aspect of the present invention. When it contains it/them, said composition can comprise one, two, three, four, five, six, seven, eight, nine, ten, eleven or all of said additional compounds selected from the group consisting of 1-chloro-1,1,2-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane.

Said azeotropic or quasi-azeotropic composition can also comprise, preferably consist of:
from 38% to 66% by weight of 1,1,2-trifluoroethane, from 34% to 62% by weight of 2-chloro-1,1,1-trifluoroethane and optionally the remainder as at least one of the additional compounds selected from group consisting of 1-chloro-1,1,2-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, the latter having a boiling point of 0° C. to 5° C. at a pressure of 1 bara;
from 38% to 66% by weight of 1,1,2-trifluoroethane, from 34% to 62% by weight of 2-chloro-1,1,1-trifluoroethane and optionally the remainder as at least one of the additional compounds selected from group consisting of 1-chloro-1,1,2-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane based on the total weight of said composition, the latter having a boiling point of 0° C. to 5° C. at a pressure of 1.013 bara;
from 50% to 94% by weight of 1,1,2-trifluoroethane, from 6% to 50% by weight of 2-chloro-1,1,1-trifluoroethane and optionally the remainder as at least one of the additional compounds selected from the group consisting of 1-chloro-1,1,2-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, the latter having a boiling point of 18° C. to 23° C. at a pressure of 2 bara;
from 72% to 99.99% by weight of 1,1,2-trifluoroethane, from 0.01% to 28% by weight of 2-chloro-1,1,1-trifluoroethane and optionally the remainder as at least one of the additional compounds selected from the group consisting of 1-chloro-1,1,2-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, the latter having a boiling point of 31° C. to 35° C. at a pressure of 3 bara.

In a particularly preferred way, the present invention provides azeotropic compositions comprising, preferably consisting of, from 50.5% to 51.5% by weight of 1,1,2-trifluoroethane and from 48.5% to 49.5% by weight of 2-chloro-1,1,1-trifluoroethane, based on the total weight of said composition, and having a boiling point of 2.5° C. to 3.5° C. at a pressure of 1 bara. In a particularly preferred way, the present invention provides azeotropic compositions comprising, preferably consisting of, from 50.5% to 51.5% by weight of 1,1,2-trifluoroethane, from 48.5% to 49.5% by weight of 2-chloro-1,1,1-trifluoroethane and optionally the remainder as at least one of the additional compounds selected from the group consisting of 1-chloro-1,1,2-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, and having a boiling point of 2.5° C. to 3.5° C. at a pressure of 1 bara.

More particularly preferably, said azeotropic composition consists of 50.9% by weight of 1,1,2-trifluoroethane and 49.1% by weight of 2-chloro-1,1,1-trifluoroethane, based on the total weight of said composition, and has a boiling point of 2.7° C. at a pressure of 1 bara.

In a particularly preferred way, the present invention provides azeotropic compositions comprising, preferably consisting of, from 51% to 52% by weight of 1,1,2-trifluoroethane and from 48% to 49% by weight of 2-chloro-1,1,1-trifluoroethane, based on the total weight of said composition, and having a boiling point of 2.5° C. to 3.5° C. at a pressure of 1.013 bara.

In a particularly preferred way, the present invention provides azeotropic compositions comprising, preferably consisting of, from 51% to 52% by weight of 1,1,2-trifluoroethane, from 48% to 49% by weight of 2-chloro-1,1,1-trifluoroethane and optionally the remainder as at least one of the additional compounds selected from the group consisting of 1-chloro-1,1,2-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, and having a boiling point of 2.5° C. to 3.5° C. at a pressure of 1.013 bara.

More particularly preferably, said azeotropic composition consists of 51.2% by weight of 1,1,2-trifluoroethane and 48.8% by weight of 2-chloro-1,1,1-trifluoroethane, based on the total weight of said composition, and has a boiling point of 3.1° C. at a pressure of 1.013 bara.

In a particularly preferred way, the present invention provides an azeotropic composition comprising, preferably consisting of, from 72.5% to 73.5% by weight of 1,1,2-trifluoroethane and from 26.5% to 27.5% by weight of 2-chloro-1,1,1-trifluoroethane, based on the total weight of said composition, and having a boiling point of 21° C. to 22° C. at a pressure of 2 bara.

In a particularly preferred way, the present invention provides an azeotropic composition comprising, preferably consisting of, from 72.5% to 73.5% by weight of 1,1,2-trifluoroethane, from 26.5% to 27.5% by weight of 2-chloro-1,1,1-trifluoroethane and optionally the remainder as at least one of the additional compounds selected from the group consisting of 1-chloro-1,1,2-trifluoroethane, 2-chloro-1,1,2-trifluoroethane, chlorotrifluoroethylene, trifluoroethylene, HF,

HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, and having a boiling point of 21° ° C. to 22° C. at a pressure of 2 bara.

More particularly preferably, said azeotropic composition consists of 73% by weight of 1,1,2-trifluoroethane and 27% by weight of 2-chloro-1,1,1-trifluoroethane, based on the total weight of said composition, and has a boiling point of 21.5° C. at a pressure of 2 bara.

FIG. 4 represents the change in the bubble point and in the dew point as a function of the content by weight of 1,1,2-trifluoroethane for the 1,1,2-trifluoroethane/2-chloro-1,1,1-trifluoroethane binary mixture at a pressure of 1 to 6 bara.

The presence of azeotropic or quasi-azeotropic compositions as defined in the present invention is confirmed by the data resulting from [FIG. 3] and [FIG. 4]. The relative volatilities of each of the compositions presented in FIGS. 3 and 4 were calculated at the liquid-vapor equilibrium.

According to another preferred embodiment, said quasi-azeotropic composition comprises, preferably consists of, from 0.01% to 0.06% by weight of 1,1,2-trifluoroethane and from 99.94% to 99.99% by weight of 2-chloro-1,1,1-trifluoroethane, based on the total weight of said composition. Preferably, said quasi-azeotropic composition comprises, preferably consists of, from 0.01% to 0.06% by weight of 1,1,2-trifluoroethane and from 99.94% to 99.99% by weight of 2-chloro-1,1,1-trifluoroethane, based on the total weight of said composition, and has a boiling point of 62° C. to 66° C. at a pressure of 6 bara. In particular, said quasi-azeotropic composition comprises, preferably consists of, from 0.01% to 0.06% by weight of 1,1,2-trifluoroethane, from 99.94% to 99.99% by weight of 2-chloro-1,1,1-trifluoroethane and optionally the remainder as at least one of the additional compounds selected from the group consisting of 1-chloro-1,1,2-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, and has a boiling point of 62° C. to 66° ° C. at a pressure of 6 bara. More particularly, said quasi-azeotropic composition comprises, preferably consists of, from 0.01% to 0.06% by weight of 1,1,2-trifluoroethane, from 99.94% to 99.99% by weight of 2-chloro-1,1,1-trifluoroethane and optionally the remainder as at least one of the additional compounds selected from the group consisting of 1-chloro-1,1,2-trifluoroethane, 2-chloro-1,1,2-trifluoroethane, chlorotrifluoroethylene, trifluoroethylene, HF, HCl, 1,1,1-trifluoroethane, 1,1-difluoroethane, 1,2-difluoroethane, 1,1,1,2-tetrafluoroethane, 1,1-difluoroethylene and 1,1,2,2-tetrafluoroethane, based on the total weight of said composition, and has a boiling point of 63.5° C. to 64° C. at a pressure of 6 bara.

1-9. (canceled)

10. An azeotropic or quasi-azeotropic composition comprising chlorotrifluoroethylene and at least one of the compounds selected from the group consisting of 1,1,2-trifluoroethane, 1,1-difluoroethane and a mixture of the two, characterized in that it contains at least 50% by weight of chlorotrifluoroethylene, based on the total weight of said composition.

11. The composition as claimed in claim 10, characterized in that said composition has a boiling point of between −40° C. and 40° C. at a pressure of between 0.5 bara and 8 bara.

12. The composition as claimed in claim 10, characterized in that said composition also comprises at least one of the additional compounds selected from the group consisting of 2-chloro-1,1,1-trifluoroethane, 2-chloro-1,1,2-trifluoroethane, 1-chloro-1,1,2-trifluoroethane, trifluoroethylene, HF, HCl, 1,1,1-trifluoroethane, 1,2-difluoroethane, 1,1,1,2-tetrafluoroethane, 1,1-difluoroethylene and 1,1,2,2-tetrafluoroethane.

13. The composition as claimed in claim 10, characterized in that said composition contains at most 20% by weight of 1,1,2-trifluoroethane, based on the total weight of said composition.

14. The composition as claimed in claim 10, characterized in that said composition contains at most 50% by weight of 1,1-difluoroethane, based on the total weight of said composition.

15. An azeotropic or quasi-azeotropic composition comprising 1,1,2-trifluoroethane and at least one of the compounds selected from the group consisting of 2-chloro-1,1,1-trifluoroethane, 1-chloro-1,1,2-trifluoroethane and a mixture of the two.

16. The composition as claimed in claim 15, characterized in that said composition has a boiling point of between −10° ° C. and 70° C. at a pressure of between 0.5 bara and 8 bara.

17. The composition as claimed in claim 15, characterized in that said composition contains between 35% and 99.99% by weight of 1,1,2-trifluoroethane, based on the total weight of said composition.

18. The composition as claimed in claim 15, characterized in that said composition contains at most 65% by weight of 2-chloro-1,1,1-trifluoroethane or at most 38% by weight of 1-chloro-1,1,2-trifluoroethane.
